(19) United States
(12) Patent Application Publication
(10) Pub. No.: US 2026/0295251 A1
(43) Pub. Date: Oct. 1, 2026

(54) STIMULATION SYSTEMS, ASSEMBLIES, AND METHODS FOR DELIVERING ELECTRIC STIMULATION TO A SUBJECT

(71) Applicant: Washington University, St. Louis, MO (US)

(72) Inventors: Matthew Gaidica, St. Louis, MO (US); Todd Braver, St. Louis, MO (US); ShiNung Ching, St. Louis, MO (US)

(21) Appl. No.: 19/578,107

(22) Filed: Mar. 25, 2026

Related U.S. Application Data
(60) Provisional application No. 63/778,076, filed on Mar. 26, 2025.

(51) Int. Cl. A61N 1/36; A61N 1/02

(52) U.S. Cl. CPC A61N 1/36025; A61N 1/025; A61N 1/36034

(57) ABSTRACT

A transcranial stimulation assembly for delivering transcranial electric stimulation to a subject is provided. The transcranial stimulation assembly includes a motherboard configured to communicate with an application installed on a stimulation computing device to receive a command from the application. The transcranial stimulation assembly also includes one or more daughterboards pluggable with one or more electrical connectors on the motherboard. The one or more daughterboards are configured to deliver electric stimulation to the subject according to the command.

### CROSS REFERENCE TO RELATED APPLICATIONS

This application claims the benefit of U.S. Provisional Application No. 63/778,076, filed on Mar. 26, 2025, the entire content and disclosure of which is hereby incorporated by reference herein in its entirety.

### STATEMENT REGARDING FEDERALLY SPONSORED RESEARCH & DEVELOPMENT

This invention was made with government support under MH132240 awarded by the National Institutes of Health. The government has certain rights in the invention.

### TECHNICAL FIELD

The field of the disclosure relates generally to electric stimulation, and more particularly, to systems, assemblies, and methods of electric stimulation.

### BACKGROUND

Transcranial electric stimulation (tES) is a non-invasive technique used to modulate brain activity by applying a low electrical current through the scalp. This technique is commonly employed in both research and clinical settings to influence cognitive functions, treat neurological disorders, and enhance rehabilitation efforts. tES is typically achieved by using electrodes placed on the scalp, which deliver a weak electrical current to specific brain regions. However, current systems for tES are limited in scalability, as they are often constrained by compliance voltage, preventing the achievement of full-scale stimulation specifications. Additionally, many existing devices lack modularity, restricting their ability to expand or adjust to different configurations and preventing individual channel calibration. This lack of flexibility limits the effectiveness of the technology, especially in applications requiring adaptability for diverse research or clinical needs. Accordingly, there is a need for improved tES systems.

This section is intended to introduce the reader to various aspects of art that may be related to various aspects of the present disclosure described or claimed below. This description is believed to be helpful in providing the reader with background information to facilitate a better understanding of the various aspects of the present disclosure. Accordingly, it should be understood that these statements are to be read in this light and not as admissions of prior art.

### BRIEF DESCRIPTION

In one aspect, a transcranial stimulation system for delivering transcranial electric stimulation to a subject is provided. The transcranial stimulation system includes a transcranial stimulation assembly including a motherboard configured to communicate with an application installed on a stimulation computing device to receive a command of electric stimulation from the application. The transcranial stimulation assembly also includes one or more daughterboards pluggable with one or more electrical connectors on the motherboard, the one or more daughterboards configured to deliver electric stimulation to a subject according to the command. The transcranial stimulation system further includes the stimulation computing device including at least one processor in communication with at least one memory device, the at least one processor programmed to receive the command from a user, the command including at least one of a waveform, a function, or a type of the electric stimulation; and communicate the command to a transcranial stimulation assembly.

In another aspect, a transcranial stimulation assembly for delivering transcranial electric stimulation to a subject is provided. The transcranial stimulation assembly includes a motherboard configured to communicate with an application installed on a stimulation computing device to receive a command from the application. The transcranial stimulation assembly also includes one or more daughterboards pluggable with one or more electrical connectors on the motherboard. The one or more daughterboards are configured to deliver electric stimulation to the subject according to the command.

In one more aspect, one or more non-transitory machine-readable storage media for delivering transcranial electric stimulation to a subject are provided. The one or more non-transitory machine-readable storage media includes a plurality of instructions stored thereon. In response to being executed, the instructions cause a system to receive a command of electric stimulation from a user. The command includes at least one of a waveform, a function, or a type of the electric stimulation. The instructions also cause the system to communicate the command to a transcranial stimulation assembly.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated examples may be incorporated into any of the above-described aspects, alone or in any combination.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a block diagram of an example transcranial stimulation system.

FIG. 2 is a schematic diagram of an example transcranial stimulation assembly of the transcranial stimulation system shown in FIG. 1.

FIG. 3A is a perspective view of an example transcranial stimulation assembly.

FIG. 3B is another perspective view of the transcranial stimulation assembly shown in FIG. 3A.

FIG. 3C is one more perspective view of the transcranial stimulation assembly shown in FIG. 3A.

FIG. 4A is a schematic circuit diagram of an example motherboard of the transcranial stimulation assembly shown in FIG. 3A.

FIG. 4B is a schematic diagram of an example print circuit board (PCB) of the motherboard shown in FIG. 4A.

FIG. 5A is a schematic circuit diagram of an example daughterboard of the transcranial stimulation assembly shown in FIG. 3A.

FIG. 5B is a schematic diagram of an example PCB of the daughterboard shown in FIG. 5A.

FIG. 6 shows screenshots of an example application on a stimulation computing device in the transcranial stimulation system shown in FIG. 1.

FIG. 7 is a flow chart of an example method for delivering transcranial electric stimulation to a subject.

FIG. 8 is a comparison of features of the transcranial stimulation system described herein with at least some known solutions.

FIG. 9 is a table of description of at least some known solutions.

FIG. 10 is a block diagram of an example user computing device.

FIG. 11 is a block diagram of an example server computing device.

FIGS. 12A and 12B show responses from stimulation.

FIG. 13 show responses of a subject from the same stimulation paradigm on separate days.

Corresponding reference characters indicate corresponding parts throughout the several views of the drawings. Although specific features of various examples may be shown in some drawings and not in others, this is for convenience only. Any feature of any drawing may be referenced or claimed in combination with any feature of any other drawing. The drawings are not to scale unless otherwise noted. Unless otherwise indicated, the drawings provided herein are meant to illustrate features of embodiments of the disclosure. These features are believed to be applicable in a wide variety of systems including one or more embodiments of the disclosure. As such, the drawings are not meant to include all conventional features known by those of ordinary skill in the art to be required for the practice of the embodiments disclosed herein.

### DETAILED DESCRIPTION

The following detailed description and examples set forth preferred materials, components, and procedures used in accordance with the present disclosure. This description and these examples, however, are provided by way of illustration only, and nothing therein shall be deemed to be a limitation upon the overall scope of the present disclosure.

The disclosed systems and methods are described, for clarity, using certain terminology when referring to and describing relevant components within the disclosure. Where possible, common industry terminology is employed in a manner consistent with its accepted meaning. Unless otherwise stated, such terminology should be given a broad interpretation consistent with the context of the present application and the scope of the appended claims.

Systems, assemblies, and methods for delivering electrical simulation to a subject is provided. As used herein, a subject may be a human, an animal, one or more parts of a human, one or more parts of an animal, or a phantom (e.g., a synthetic model used for testing). Transcranial electrical stimulation is described herein as examples for illustration purposes only. Systems, assemblies, and methods described herein may be used to apply electrical stimulation to other parts of the subject, such as muscle.

Traditional transcranial electric stimulation (tES) systems are often limited by fixed configurations, lack of scalability, and insufficient flexibility for adapting to diverse research or clinical needs. These systems typically offer a limited number of stimulation channels, lack modularity, and do not support dynamic customization of parameters, resulting in inefficiencies or compromised performance in various scenarios. Furthermore, existing systems struggle with voltage compliance and safety, as they are not equipped to handle the wide variability in impedance during stimulation. Accordingly, there is a need for a tES system that provides scalable, modular, and adaptable functionality, robust power management, and precise control over stimulation parameters for various use cases, to meet the demands of research and clinical environments.

The present disclosure addresses these challenges with a modular transcranial stimulation assembly including a motherboard and one or more daughterboards. Each daughterboard is configured to independently deliver electric stimulation, allowing the system to scale from a single channel to multiple channels. The motherboard manages communication with external devices and power distribution while ensuring each daughterboard operates as intended. For example, the motherboard detects that each daughterboard is operating via voltage or current signals, allowing for seamless integration and real-time feedback on system configuration.

The system may further include power management capabilities, where the motherboard optimizes power consumption by monitoring current usage from each daughterboard. This ensures that the system operates within thresholds while maintaining performance. The motherboard may also communicate with an external stimulation computing device via wireless or wired communication, enabling remote control of stimulation parameters such as waveform, frequency, and intensity through an application or computing interface. Additionally, the modular design enables independent calibration of each daughterboard, providing stimulation tailored to individual needs, while a current-limiting feature increases safety by preventing over-stimulation.

The present disclosure provides a scalable, flexible, and cost-effective solution for non-invasive brain stimulation. The system's modularity allows for easy expansion or reduction in the number of stimulation channels, making it adaptable to a wide range of research and clinical protocols. By offering high compliance voltage, dynamic power management, and independent calibration, the system ensures optimal stimulation performance while prioritizing user safety. With its ability to accommodate various configurations and requirements, the system meets the evolving needs of both researchers and clinicians in the field of tES.

The present disclosure addresses several key design challenges for a portable brain stimulation device. The transcranial stimulation system includes multiple constraints particularly focusing on isolation, form factor, and the need for a compact, yet fully functional solution. The transcranial stimulation system includes stimulation channels, ensuring that the stimulation may be accurately targeted without interference between channels. The transcranial stimulation system architecture includes isolation techniques implemented to protect subjects from potential electrical hazards. This isolation had been achieved by creating physical gaps between certain components, limiting that voltage spikes or disturbances in the power supply from affecting the stimulation side of the system.

Additionally, the transcranial stimulation system may be equipped with a command interface accessible via an application or a computer, both capable of adjusting settings, monitoring stimulation parameters, and controlling the device's operation. These features facilitate integration of the transcranial stimulation system into various setups and users to maintain control and monitor the system remotely.

The transcranial stimulation system may be use in research, clinical, and/or home uses involving brain stimulation, offering a portable alternative to conventional systems, with features optimized for ease of use, safety, and adaptability in dynamic environments.

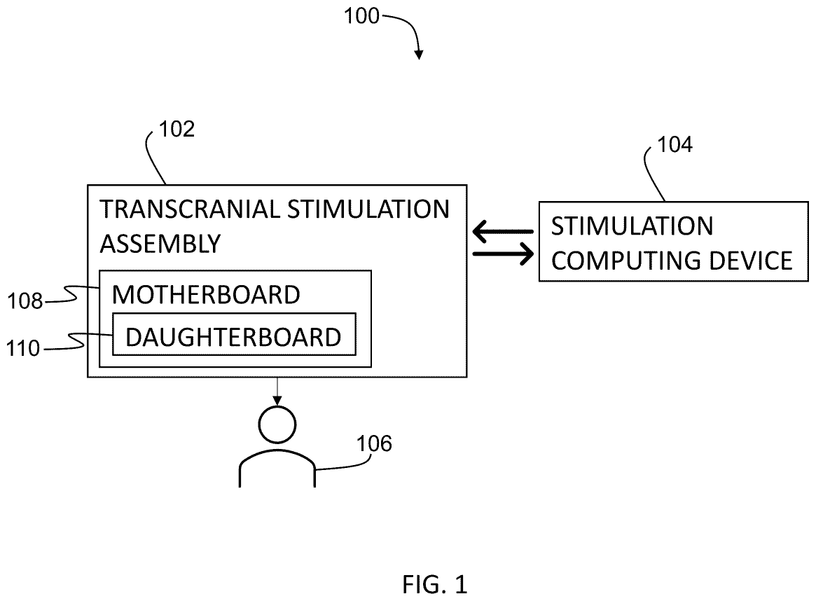

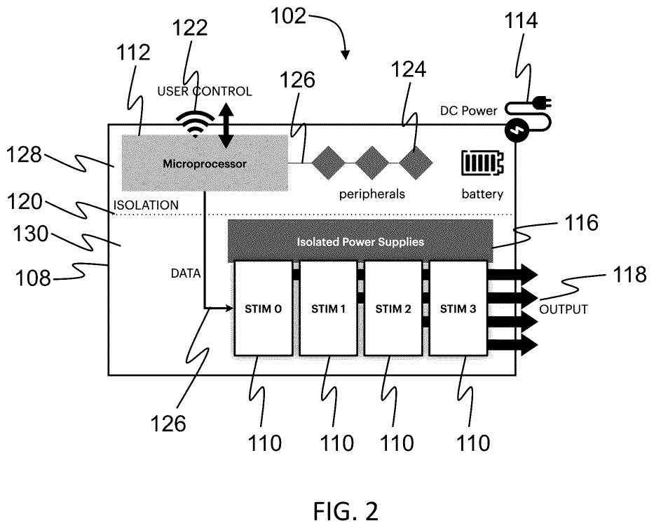

FIG. 1 is a block diagram of an example transcranial stimulation system 100. FIG. 2 is a schematic diagram of an example transcranial stimulation assembly 102 of the transcranial stimulation system 100 shown in FIG. 1.

In the example embodiment, the transcranial stimulation system 100 includes a transcranial stimulation assembly 102 and a stimulation computing device 104. The transcranial stimulation assembly 102 is a device designed for the non-invasive delivery of electrical stimulation 118 to a subject 106 through electrodes placed on the subject 106. The transcranial stimulation assembly 102 is configured to generate and regulate electrical currents to ensure that parameters such as current intensity, duration, waveform, and frequency comply with requirements stipulated by standards and/or health organizations. In operation, the current is low intensity (in the ranges of milliamps), and the stimulation duration may range from several minutes to hours. The electrical stimulation 118 may be adapted to meet the specific needs of different clinical or research protocols. In addition, the transcranial stimulation assembly 102 may include safety features to monitor and ensure that the stimulation process is functioning properly throughout the duration of application of the stimulation.

In the example embodiment, the transcranial stimulation assembly 102 utilizes electrodes placed on specific locations on the scalp of the subject 106. These locations are chosen depending on the target brain regions for stimulation, which may vary according to clinical or research protocols. The electrodes are constructed from conductive materials that facilitate delivery of electrical currents. The transcranial stimulation assembly 102 may include anodal and cathodal electrodes: the anodal electrode is used to stimulate neural activity, while the cathodal electrode is used to inhibit neural activity. In some embodiments, multiple electrodes may be used simultaneously or sequentially to target different brain regions or to create complex stimulation patterns.

In the example embodiment, to enable the scalability of the transcranial stimulation assembly 102 (see FIG. 2), the transcranial stimulation assembly 102 includes a motherboard 108 and one or more daughterboards 110. The main functions of the transcranial stimulation assembly 102, such as providing power supply, signal generation and processing, and general input and output, are implemented by the motherboard 108. A system bus 126 is provided to communicate with and supply power to the daughterboards 110. Systems and methods described herein are advantageous in enabling scalability and providing flexible and convenient solutions to a user because the user may have different needs at different times. When a different need arises, a completely new design of circuitry is unnecessary. Instead, a daughterboard 110 that meets the need is used by plugging the daughterboard 110 into the motherboard 108.

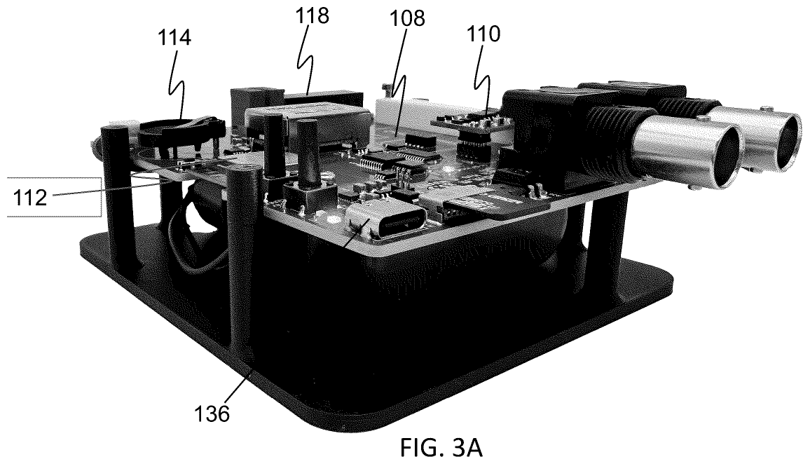

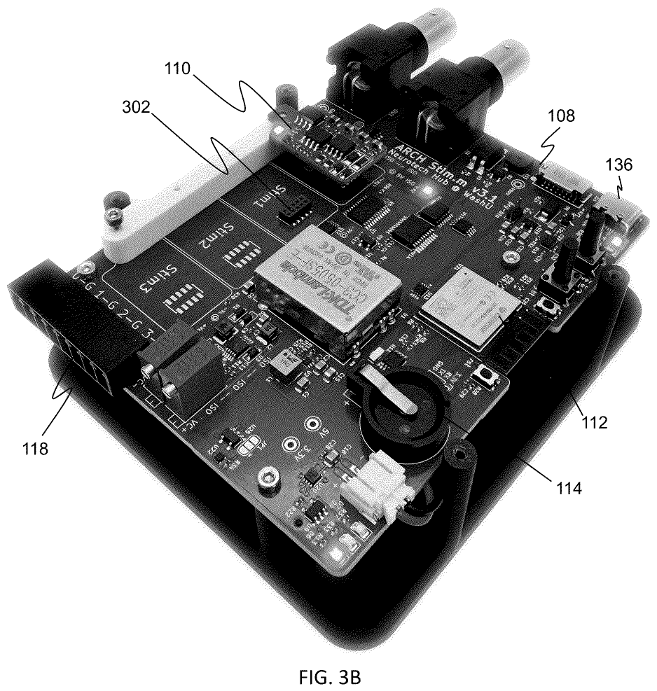

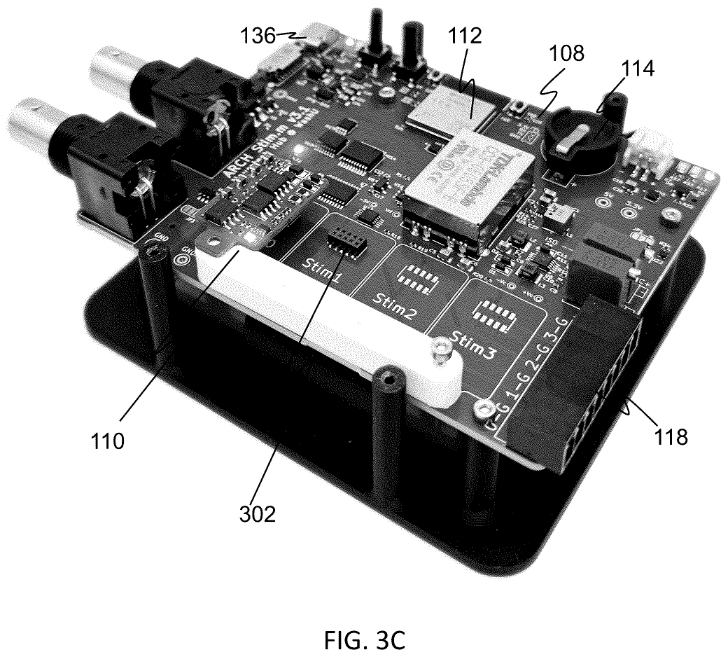

In the example embodiment, the transcranial stimulation assembly 102 is modular, designed for scalability to meet the varying needs of different users. This modular architecture facilitates the customization of the system for different stimulation protocols and supports scalability from single-channel to multi-channel systems. To add a channel, a daughterboard 110 is plugged into one or more electrical connectors 302 (see FIGS. 3B and 3C, described later), such as female connectors and/or male connectors, on the motherboard 108, and establish electrical connection with the motherboard 108. To remove or replace a channel, the daughterboard 110 to be removed or replaced is disconnected from the electrical connectors 302, such as by pulling the daughterboard 110 apart from the electrical connectors 302.

In the example embodiment, the motherboard 108 includes a microprocessor 112, a power supply 114, isolated power supply 144 or voltage rails 116, an isolation boundary 120, a communication interface 122, and one or more peripheral devices 124. The power supply 114 may be a battery and/or a connector to a power supply. The connector may be a universal serial bus (USB) connector. A USB connector is advantageous in simplifying the design, assembling, and connection of the transcranial stimulation assembly 102 by enabling transmitting power and signals via the single USB connection.

In the example embodiment, the microprocessor 112 includes a processor-based microcontroller including a processor and a memory device wherein executable instructions, commands, and control algorithms, as well as other data and information needed to operate the motherboard 108, are stored. The memory device may be, for example, a random access memory (RAM), and other forms of memory used in conjunction with RAM memory, including but not limited to flash memory (FLASH), programmable read only memory (PROM), and electronically erasable programmable read only memory (EEPROM).

As used herein, the term “processor-based” microcontroller refers not only to controller devices including a processor or microprocessor as shown, but also to other equivalent elements such as microcomputers, programmable logic controllers, reduced instruction set circuits (RISC), application specific integrated circuits and other programmable circuits, logic circuits, equivalents thereof, and any other circuit or processor capable of executing the functions described below. The processor-based devices listed above are examples only, and are thus not intended to limit in any way the definition and/or meaning of the term “processor-based.”

In the example embodiment, the microprocessor 112 uses executable instructions stored in the memory device to manage the parameters of the electrical current delivered to the subject 106. The microprocessor 112 is coupled to the communication interface 122, one or more peripherals 124, and the daughterboards 110 via a system bus 126. The microprocessor 112 communicates with the stimulation computing device 104 through the communication interface 122 to receive stimulation instructions and adjust system parameters accordingly. The motherboard 108 supports wireless communication, such as Wi-Fi or Bluetooth Low Energy (BLE), which enables remote monitoring and controlling of the transcranial stimulation assembly 102.

In some embodiments, the microprocessor 112 is designed to accommodate different programming models or microcontroller architectures based on the specific requirements of the transcranial stimulation system 100, such as requirements on power efficiency or processing speed. The microprocessor 112 may also include a feedback mechanism that adjusts the stimulation parameters dynamically based on real-time measurements from the electrodes, enabling adaptive stimulation to meet specific clinical or research needs.

In the example embodiment, the motherboard 108 isolates the power supply 114, coordinates various system operations via the microprocessor 112, and includes precision analog circuits for controlling stimulation voltages. In some embodiments, the motherboard 108 may include dual power supply rails 116, e.g., ±36 V, to support bipolar stimulation and ensure reliable, adequate current delivery under varying impedance conditions across subjects, thereby maintaining the quality of the electrical stimulation under varying impedance conditions of the subject 106.

In some embodiments, high voltage compliance rails 116 may be utilized to enable current delivery under varying impedance conditions. These bipolar compliance rails, typically set at ±36 V, facilitate that the transcranial stimulation assembly 102 delivers sufficient current even when the impedance of the subject's skin or scalp changes.

In the example embodiment, the daughterboards 110 are responsible for delivering electrical stimulation through individual stimulation channels. The daughterboards 110 may control one channel and includes features, such as tunable biphasic stimulation and active current sensing. The daughterboard 110 may include current-limiting features for safety. The modular nature of the transcranial stimulation assembly 102 facilitates flexibility in the number of channels by adding or removing the daughterboards 110 as needed, which enables customization of the system for specific applications. In some embodiments, the daughterboards 110 is configured to handle multiple channels per board, offering an increased degree of integration for multi-channel systems.

In the example embodiment, communication between the motherboard 108 and the daughterboards 110 takes place over an isolated SPI interface, which reduces the risk of electrical interference or accidental shocks. This isolated interface ensures that control signals and feedback from the stimulation process are transmitted safely, and prevents electrical disruption.

In the example embodiment, the transcranial stimulation system 100 may be used with multiple daughterboards 110, with each daughterboard being calibrated independently, enabling adjustments on a per-channel basis. This modular approach facilitates easy reconfiguration of the transcranial stimulation system 100 to accommodate different protocols and supports scaling from a single-channel to a multi-channel system.

In some embodiments, the motherboard 108 may include a different number of daughterboard connectors based on the intended application. For example, an alternative embodiment may have a motherboard with eight or more connectors, instead of four connectors as shown in FIG. 2. The ability to include multiple daughterboards 110 ensures that the transcranial stimulation system 100 can scale to meet a variety of stimulation requirements while maintaining independent control over each stimulation channel.

In some embodiments, the transcranial stimulation system 100 also includes independent hardware control for each stimulation channel. This fine-tuned control enables precise adjustments on a per-channel basis, for managing stimulation across multiple regions of the brain. By offering independent control, the system reduces the risk of unintended overlap or over-stimulation between channels and ensures that each stimulation channel is customized to meet the specific needs of the subject.

In some embodiments, a current shunt is implemented in each daughterboard 110 to limit the electrical current delivered to the subject 106 and to comply with the safe requirements for the subject 106. This feature limits the current from exceeding safe, pre-defined thresholds, adding an additional layer of protection against overcurrent situations.

In the example embodiment, the transcranial stimulation system 100 may also incorporate a real-time feedback mechanism, which monitors the stimulation parameters and adjusts them as necessary to enable safe and effective operation. This feedback system measures the current and impedance, and enables real-time adjustments to maintain the optimal stimulation conditions, thereby reducing or preventing issues related to unsafe stimulation levels.

One potential application for the transcranial stimulation system 100 is as a research platform intended for academic and institutional users. In addition, as multi-channel brain stimulation techniques gain prominence in emerging areas like brain-computer interfaces (BCIs), the transcranial stimulation system 100 may be adapted for use in consumer applications such as transcranial Direct Current Stimulation (tDCS), transcranial Alternating Current Stimulation (tACS), or transcranial Random Noise Stimulation (tRNS). The modular design of the transcranial stimulation system 100 makes it versatile, allowing for cost-effective scaling and customization to meet the evolving needs of both researchers and consumers.

In the example embodiments, the transcranial stimulation system 100 is designed for scalability and economic feasibility. Initially targeted at academic research, the system can be mass-produced at an affordable price. The modular design ensures that each system can be independently calibrated, making it a versatile and cost-effective solution for both research and consumer applications.

FIG. 3A is a perspective view of an example transcranial stimulation assembly. FIG. 3B is another perspective view of the transcranial stimulation assembly shown in FIG. 3A. FIG. 3C is one more perspective view of the transcranial stimulation assembly shown in FIG. 3A.

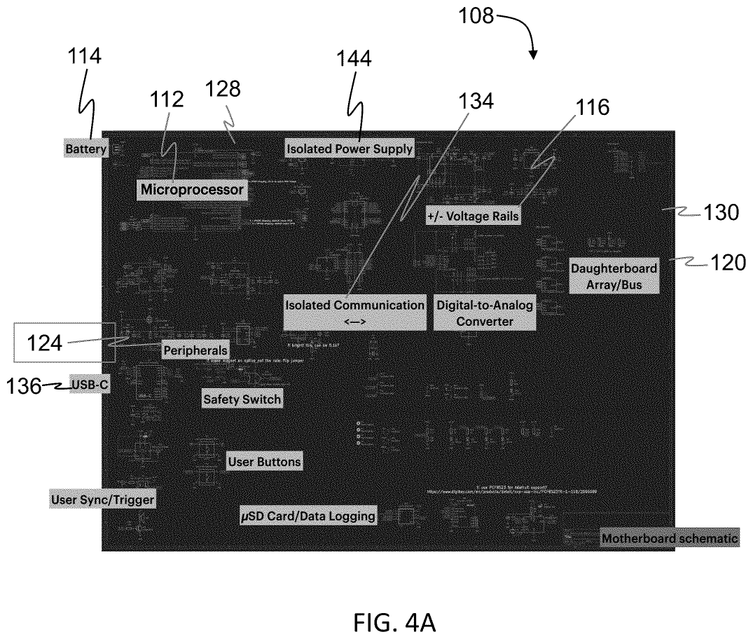

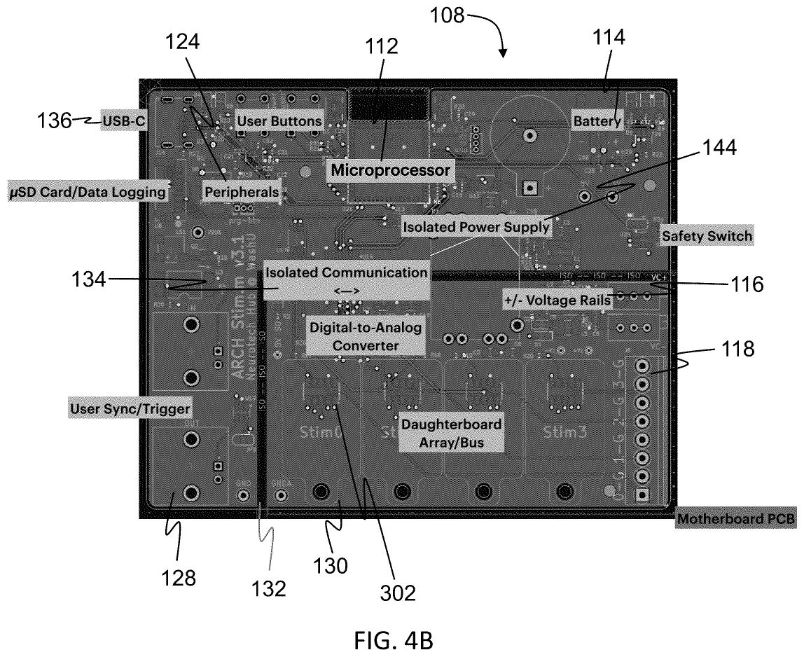

FIG. 4A is a schematic circuit diagram of an example motherboard of the transcranial stimulation assembly shown in FIG. 3A. FIG. 4B is a schematic diagram of an example print circuit board (PCB) of the motherboard shown in FIG. 4A.

In the example embodiment, the motherboard 108 includes an isolated portion 130 and a general portion 128. The isolated portion 130 is marked by a box 120 in dashed lines.

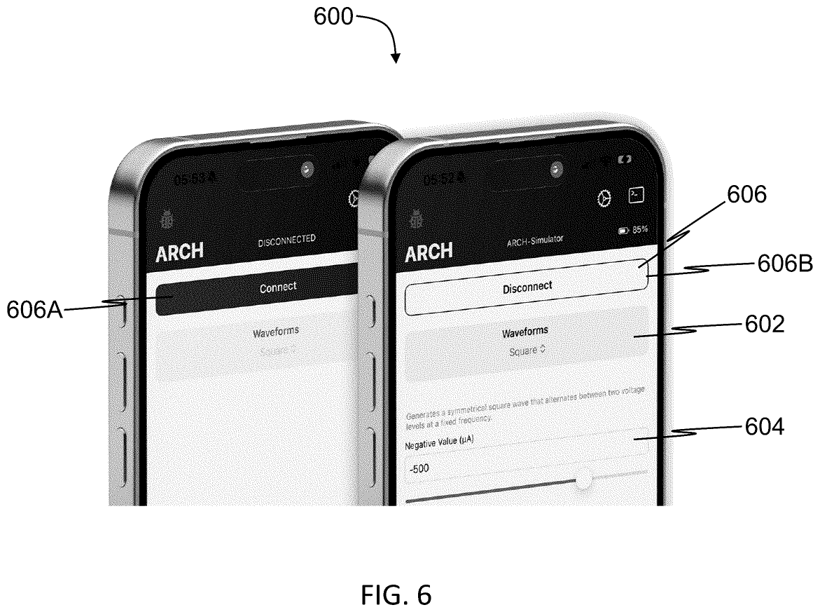

In the example embodiment, the general portion 128 includes the microprocessor 112. The microprocessor 112 includes waveform generation, an interface such as an application programming interface for interfacing with an application 600 (see FIG. 6, described later), general purpose input and output, and communication with the peripherals 124. The microprocessor 112 is also configured to detect the voltage level of a battery 114, and/or whether a universal serial bus (USB) is electrically connected.

In the example embodiment, the isolated portion 130 is configured to deliver electrical stimulation via the daughterboard 110 to the subject 106. The power supply and communication between the isolated portion 130 and the general portion 128 are isolated from one another via a physical gap 132 (see FIG. 4B) between the isolated portion 130 and the general portion 128. The only electrical connections between the isolated portion 130 and the general portion 128 are isolated communication 134 for communication between the motherboard and the daughterboards 110 and the isolated power supply 144 for the motherboard to supply power to the daughterboards 110. Because the isolated portion 130 delivers electrical stimulation to the subject 106 but the power supply 114 to the motherboard may experience power surge, such as short circuits or unstable power supply, the isolation ensures that any electrical spike that is above the safety levels for the subject is blocked from being delivered to the subject 106.

In the example embodiment, in the meantime, to bridge between the general portion 128 and the isolated portion 130, general purpose voltage levels, such as a direct current (DC) voltage of 5 V, are sent into the isolated portion 130 via the isolated power supply 144. The isolated portion 130, via the voltage rail 116, is configured to generate analog voltage levels suitable for the subject 106. Voltage delivered by the voltage rail 116 is limited to ±36 V, which is suitable for human application.

In the example embodiment, the voltage may be derived from one single USB connection 136 by the motherboard 108. The transcranial stimulation assembly 102 is desired to be portable, where the form factor of the transcranial stimulation assembly 102 is small, such as in the size of a cellular phone, and does not significantly interfere with the subject's movement, such as walking. Designing a circuit architecture capable of delivering and precisely control biphasic waveforms in the range of ±36 V based on power supply from a single USB while ensuring isolation and safe delivery to a subject within the desired form factor is challenging. Typical circuits, especially solid-state circuits suitable for precise control, that use battery power or a USB power with the desired form factor have limited voltage ranges, like 16 V or less. To achieve a voltage higher than that, the sizes of the components increase drastically and/or the current becomes too high for human application. Further, the cascading effects of power constraints from the power supply at the motherboard 108 down to individual components become challenging to manage as the complexity of the circuits and the number of daughterboards 110 increase. Form factors play another significant role because only limited numbers of amplifiers that have reduced form factors and operate in the desired voltage range, especially solid-state components, are available to meet the need of being able to control the waveforms in real time and in high resolution.

In the example embodiment, to address the above-described challenges, in designing the circuit architecture to provide desired voltages by the voltage rail 116, several considerations are included. The maximum input is derived using a single USB connector or a battery. The efficiency factors of the general portion 128 is limited such that a certain voltage is delivered across the isolation gap 132 to the voltage rail 116. The components of the circuits are each selected to meet the above-described constraints.

In some example embodiments, the motherboard 108 of the transcranial stimulation assembly 102 is configured to detect that the one or more daughterboards 110 are operating or electrically connected with the motherboard 108, based on a detection of at least one of a voltage or a current from the one or more daughterboards 110. A binary voltage detected from the daughterboards 110 may be used to indicate whether the daughterboard 110 is operating, where one of the binary values indicates operating and the other one of the binary values indicates not operating. Alternatively or additionally, a current sensed by the daughterboards 110 may be used to indicate that the daughterboard 110 is operating. The current may be compared with a threshold. For example, if the current is at or above the threshold, the daughterboard 110 is determined operating. If the current is at or below the threshold, the daughterboard 110 is determined not operating.

In the example embodiments, the motherboard 108 of transcranial stimulation assembly is configured to manage power consumption by the one or more daughterboards 110 to meet one or more requirements.

In the example embodiments, the transcranial stimulation assembly 102 includes a universal serial bus (USB) connector configured to receive communication and power from the stimulation computing device 104 via a single USB connection.

In the example embodiments, the motherboard 108 of the transcranial stimulation assembly 102 is configured to communicate with the stimulation computing device 104 via at least one of a wireless communication or a wired communication.

In the example embodiments, the motherboard 108 of the transcranial stimulation assembly 102 is configured to receive the command programmed by a user.

In the example embodiments, the motherboard 108 of the transcranial stimulation assembly 102 is configured to receive the command including at least one of a waveform, an amplitude, a function, or a type of the electric stimulation.

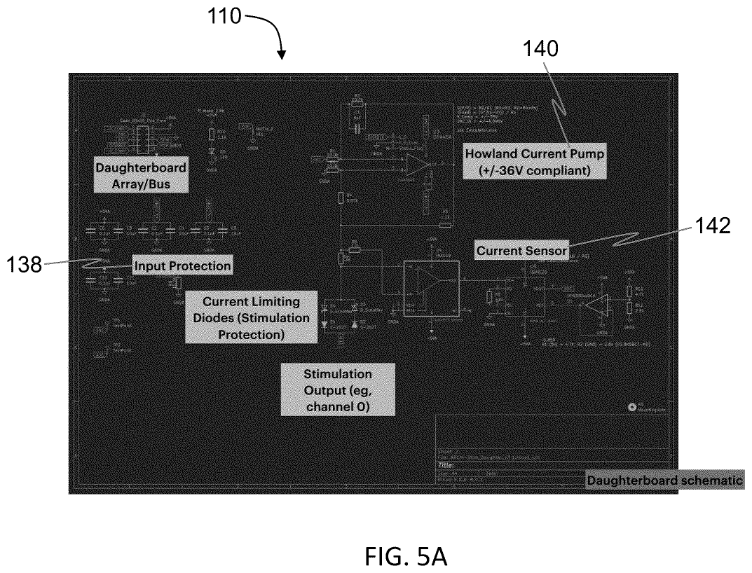

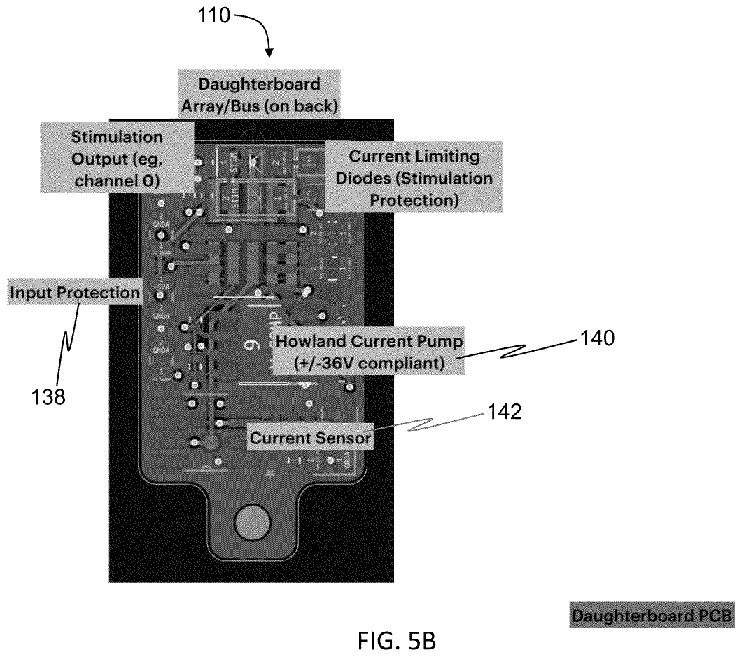

FIG. 5A is a schematic circuit diagram of an example daughterboard of the transcranial stimulation assembly shown in FIG. 3A. FIG. 5B is a schematic diagram of an example PCB of the daughterboard shown in FIG. 5A.

In the example embodiment, the daughterboard 110 includes an input protection 138 configured to filter out voltage and/or current spikes, especially during plugging into and pulling out of the motherboard. The daughterboard 110 further includes a current amplifier 140. An example current amplifier is a Howland current pump 140. The current amplifier 140 is ±36 V compliant, where the current amplifier 140 is configured to operate with the voltage being limited between ±36 V.

In the example embodiments, one or more daughterboards 110 of the transcranial stimulation assembly 102 are configured to detect a current usage by the one or more daughterboards 110 and send the current usage to the motherboard 108. The motherboard 108 is configured to manage the power consumption based on the current usage. For example, the daughterboard 110 includes a current sensor 142. The current sensor 142 is configured to detect current output from the daughterboard 110. The sensed current is provided as feedback to the motherboard 108. Based on the sensed current, the motherboard 108 determines that the daughterboard 110 is plugged in, monitors the power consumption by the daughterboard 110, and controls and adjusts the parameters of the electrical stimulation if needed. In some embodiments, the motherboard is further configured to detect the logic levels and/or analog voltages of each channel, and configured to control the power outputs to the channels such that requirements on power, current, and/or voltage delivered to the subject 106 are met while providing electrical stimulation according to the commands.

FIG. 6 shows screenshots of an example application 600 on a stimulation computing device in the transcranial stimulation system 100 shown in FIG. 1. In the example embodiment, the application 600 is shown being run on a mobile device, such as a cellular phone. In other embodiments the application 600 may be installed and run on any computer device including, but not limited to, personal computers, servers, tablets, smart televisions, smartwatches, or other. In some embodiments, the application 600 is a mobile app. In one example, the application 600 is a web-based application, where a user may access the application 600 via a computing device connected to the Internet. The application 600 facilitates a user, such as the subject 106 or a technician/an operator, to enter commands of the electrical stimulation. Example commands include waveforms 602, like square waves, and/or amplitudes 604 of the stimulation. The entry of commands may be discrete, such as by toggling through choices. The entry may be continuous, such as by moving a sliding bar. The application 600 may include other commands, such as starting or stopping the stimulation. The application 600 also includes a connect/disconnect button 606. The connect/disconnect button 606 may be toggled between disconnect 606B and connect 606A. To connect with the motherboard 108, the field of connect 606A is clicked or tapped. The commands entered by the user are transmitted to the motherboard 108. To disconnect from the motherboard 108, the field of disconnect 606B is clicked or tapped. The stimulation computing device 104 may provide options to adjust settings of the application 600. For example for the application 600, types of stimulation, like continuous or intermittent stimulation, may be selected. Continuous stimulation refers to stimulation applied by continuous electrical signals for a period of time. Intermittent stimulation refers to stimulation applied intermittently and/or by applying pulsed electrical signals.

Referring back to FIG. 1, the commands entered by a user via the stimulation computing device 104 are transmitted to the motherboard 108 of the transcranial stimulation assembly 102. The user may be the subject 106 or a technician or an operator operating the transcranial stimulation assembly 102. The microprocessor 112 is programmed to receive the commands and generate and deliver electrical stimulation based on the commands. For example, the user selects a −500 μA square wave in the application 600 (see FIG. 6). The commands of a square wave at an amplitude of −500 μA are transmitted from the transcranial stimulation assembly 102 via wireless or wired communication to the motherboard 108. The motherboard 108 is configured to deliver −500 μA square wave to the subject via the daughterboard 110 connected with electrodes coupled with the subject 106. Alternatively or additionally, data acquired by the motherboard 108, such as a successful treatment flag, may be transmitted to the stimulation computing device 104.

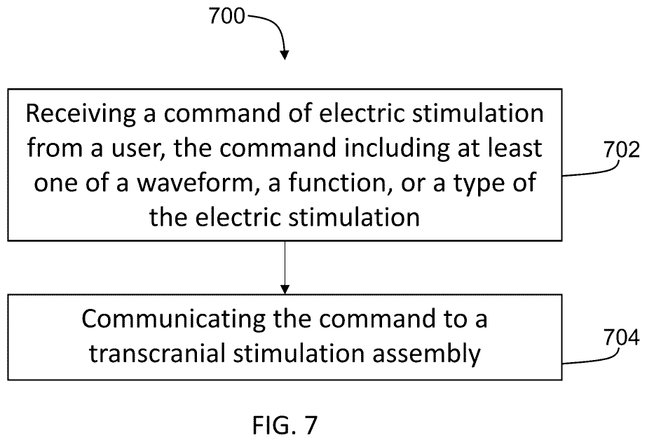

FIG. 7 is a flow chart of an example method 700 for delivering transcranial electric stimulation to a subject. The method 700 may be implemented with the motherboard 108. In the example embodiment, the method 700 includes receiving 702 a command of electric stimulation from a user. The command includes at least one of a waveform, a function, or a type of the electric stimulation. The method 700 also includes communicating 704 the command to a transcranial stimulation assembly.

In the example embodiments, the method 700 may include communicating and powering the transcranial stimulation assembly via a single universal serial bus (USB) connection. Additionally or alternatively the method 700 may include communicating with the transcranial stimulation assembly via at least one of a wireless communication or a wired communication.

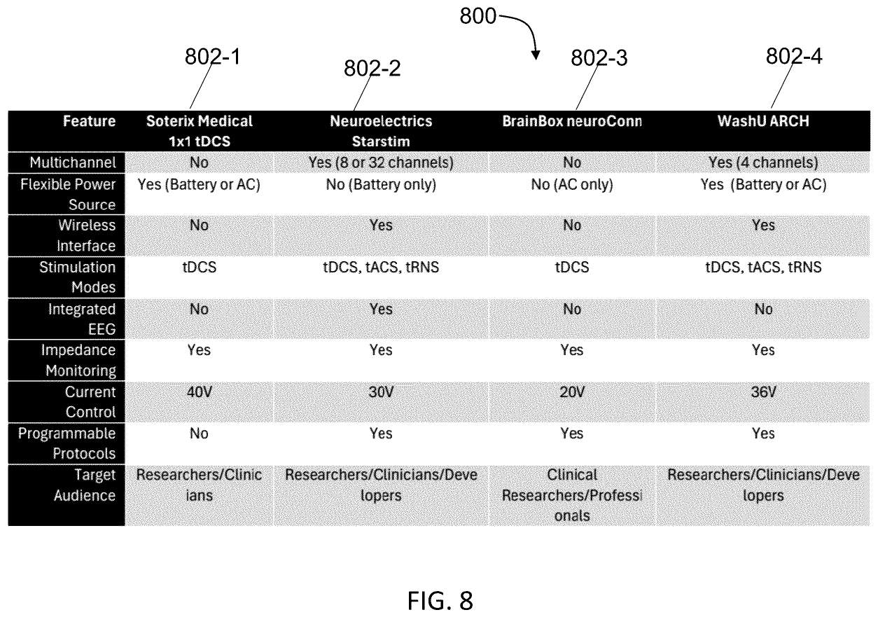

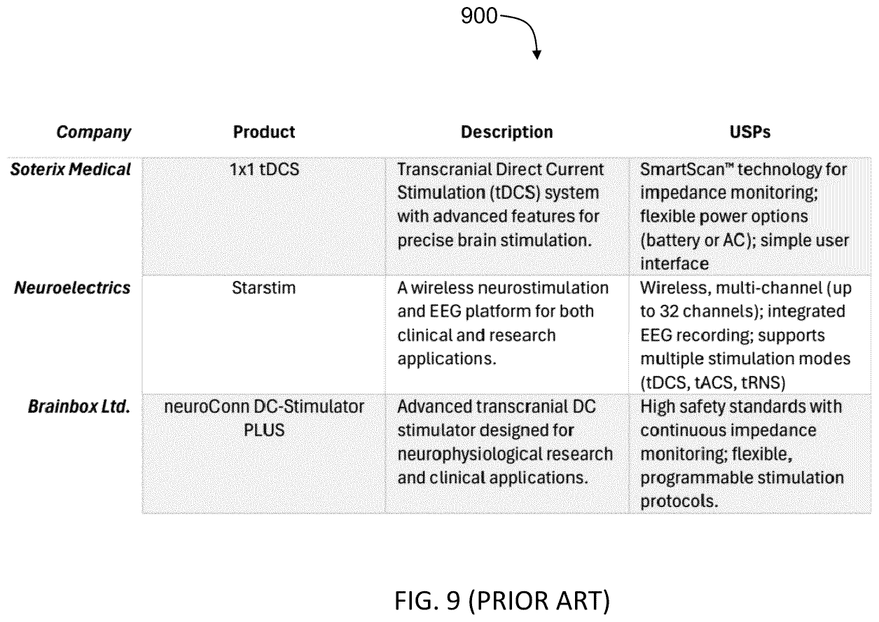

FIG. 8 is a comparison 800 of features of the transcranial stimulation system described herein with at least some known solutions. Columns 802-1, 802-2, and 802-3 show performance of known solutions, while column 802-4 show performance of the systems, assemblies, methods, and solutions described herein. FIG. 9 is a table 900 of description of at least some known solutions.

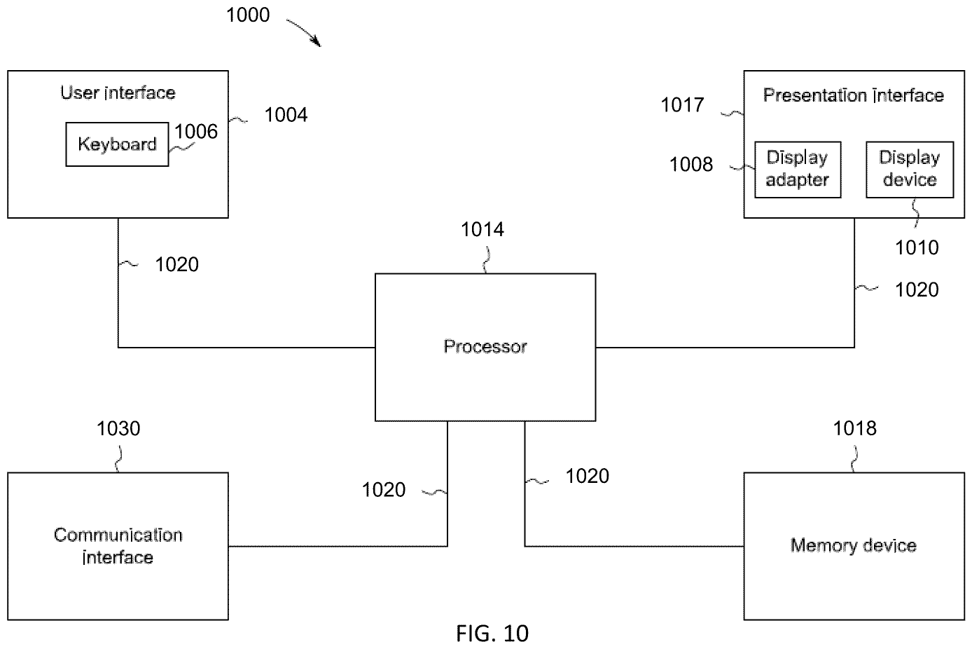

FIG. 10 is a block diagram of an example user computing device 1000. The stimulation computing device 104 may be implemented with one or more user computing devices 1000. In the exemplary embodiment, the computing device 1000 includes a user interface 1004 that receives at least one input from a user. The user interface 1004 can include a keyboard 1006 that enables the user to input pertinent information. The user interface 1004 can also include, for example, a pointing device, a mouse, a stylus, a touch sensitive panel (e.g., a touch pad and a touch screen), a gyroscope, an accelerometer, a position detector, and/or an audio input interface (e.g., including a microphone).

Moreover, in the exemplary embodiment, the computing device 1000 includes a presentation interface 1017 that presents information, such as input events and/or validation results, to the user. Presentation interface 1017 can also include a display adapter 1008 that is coupled to at least one display device 1010. More specifically, in the exemplary embodiment, the display device 1010 can be a visual display device, such as a cathode ray tube (CRT), a liquid crystal display (LCD), a light-emitting diode (LED) display, and/or an “electronic ink” display. Alternatively, the presentation interface 1017 can include an audio output device (e.g., an audio adapter and/or a speaker) and/or a printer.

The computing device 1000 also includes a processor 1014 and a memory device 1018. The processor 1014 is coupled to the user interface 1004, the presentation interface 1017, and the memory device 1018 via a system bus 1020. In the exemplary embodiment, the processor 1014 communicates with the user, such as by prompting the user via the presentation interface 1017 and/or by receiving user inputs via the user interface 1004. The term “processor” refers generally to any programmable system including systems and microcontrollers, RISC, CISC, ASIC, PLC, and any other circuit or processor capable of executing the functions described herein. The above examples are exemplary only, and thus are not intended to limit in any way the definition and/or meaning of the term “processor.”

In the example embodiment, the memory device 1018 includes one or more devices that enable information, such as executable instructions and/or other data, to be stored and retrieved. Moreover, the memory device 1018 includes one or more computer readable media, such as, without limitation, DRAM, SRAM, a solid state disk, and/or a hard disk. In the exemplary embodiment, the memory device 1018 stores, without limitation, application source code, application object code, configuration data, additional input events, application states, assertion statements, validation results, and/or any other type of data. The computing device 1000, in the exemplary embodiment, can also include a communication interface 1030 that is coupled to the processor 1014 via the system bus 1020. Moreover, the communication interface 1030 is communicatively coupled to data acquisition devices.

In the exemplary embodiment, the processor 1014 can be programmed by encoding an operation using one or more executable instructions and providing the executable instructions in the memory device 1018. In the exemplary embodiment, the processor 1014 is programmed to select a plurality of measurements that are received from data acquisition devices.

In operation, a computer executes computer-executable instructions embodied in one or more computer-executable components stored on one or more computer-readable media to implement aspects of the invention described and/or illustrated herein. The order of execution or performance of the operations in embodiments of the invention illustrated and described herein is not essential, unless otherwise specified. That is, the operations can be performed in any order, unless otherwise specified, and embodiments of the invention can include additional or fewer operations than those disclosed herein. For example, it is contemplated that executing or performing a particular operation before, contemporaneously with, or after another operation is within the scope of aspects of the invention.

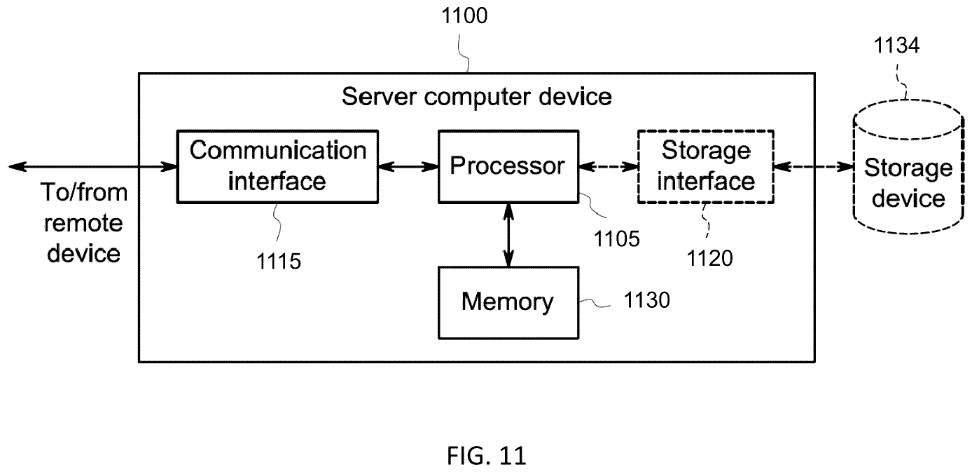

FIG. 11 is a block diagram of an example server computing device 1100. The stimulation computing device 104 may be implemented with one or more server computing devices 1100. The server computer device 1100 also includes a processor 1105 for executing instructions. Instructions may be stored in a memory device 1130, for example. The processor 1105 may include one or more processing units (e.g., in a multi-core configuration).

In the example embodiment, the processor 1105 is operatively coupled to a communication interface 1115 such that the server computer device 1100 is capable of communicating with a remote device or another server computer device 1100. For example, the communication interface 1115 may send data to the transcranial stimulation assembly 102 via the Internet or wireless communication.

In the example embodiment, the processor 1105 may also be operatively coupled to a storage device 1134. The storage device 1134 is any computer-operated hardware suitable for storing and/or retrieving data. In some embodiments, the storage device 1134 is integrated in the server computer device 1100. For example, the server computer device 1100 may include one or more hard disk drives as storage device 1134. In other embodiments, the storage device 1134 is external to the server computer device 1100 and may be accessed by a plurality of server computer devices 1100. For example, the storage device 1134 may include multiple storage units such as hard disks and/or solid-state disks in a redundant array of independent disks (RAID) configuration. The storage device 1134 may include a storage area network (SAN) and/or a network attached storage (NAS) system.

In some embodiments, the processor 1105 is operatively coupled to the storage device 1134 via a storage interface 1120. The storage interface 1120 is any component capable of providing the processor 1105 with access to the storage device 1134. The storage interface 1120 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing the processor 1105 with access to the storage device 1134.

In some embodiments, the stimulation computing device 104 is implemented with a combination of the server computing device 1100 and the user computer device 1000.

### EXAMPLE

To demonstrate capabilities of the neurostimulation device and model-based stimulation strategy, repeated stimulation experiments were performed in a group of 5 participants with repeated sessions for some participants. Using the device's real-time programming capabilities, stimulation pulses were delivered in which the duration, amplitude, and polarity were resampled in real time to test a wide range of parameters for each participant. The modular design was leveraged to alter which brain areas were targeted using three electrode configurations for comparison. Stimulation electrodes were directly integrated into the EEG-recording cap using custom-designed holders that carry electrolytic gel to produce electrical contact with the skin.

### Demonstration Criteria

The initial demonstration was designed to validate the following capabilities:
1) Integration of simultaneous brain stimulation and recording (EEG)
2) Elicitation of an immediate stimulation-evoked brain response that can be controlled through stimulation parameters.
3) Person-specificity and stability over-time of the evoked response.

### Demonstration of Concept Results

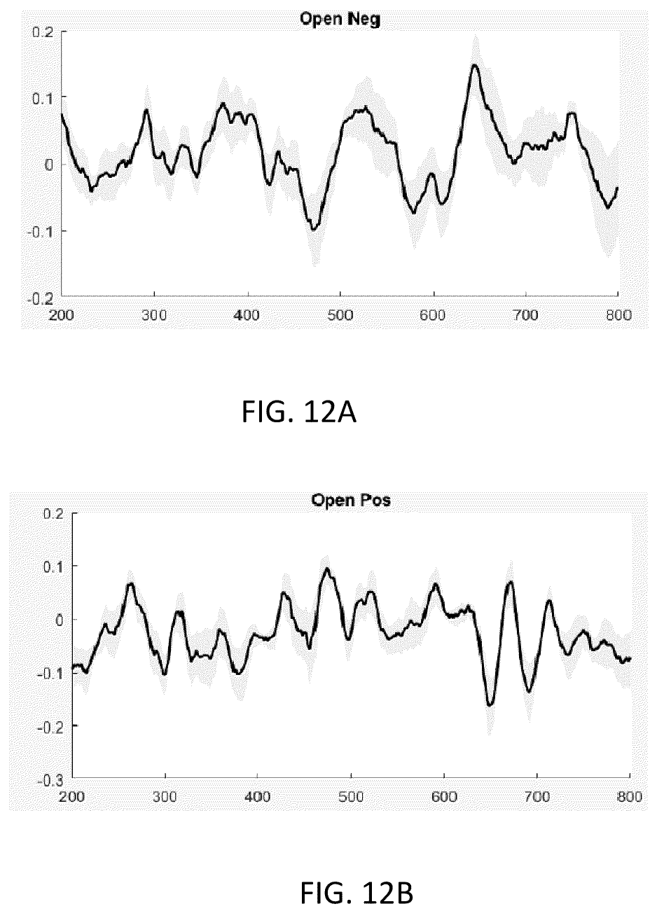

Results demonstrate the generation of a statistically-significant changes in synchronized brain activity immediately after stimulation. The type of brain rhythm promoted depended upon the direction of electrical current flow. Data below shows that anodal (“negative”) stimulation generated an immediate alpha-band (10 Hz, FIG. 12A) response whereas cathodal (“positive”) stimulation generated a response in the faster beta (20 Hz, FIG. 12B) band. These subsecond responses are impossible to observe without the ability of the device described herein to instantaneously turn stimulation on/off and programmatically test stimulation settings.

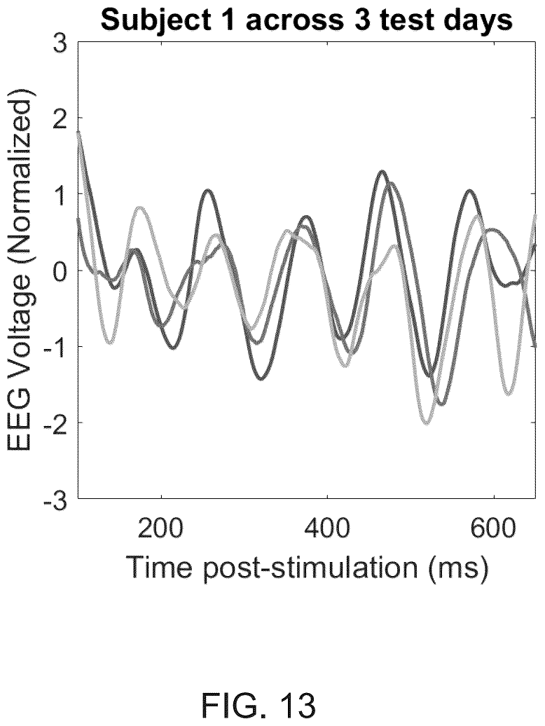

For one of the subjects, the same stimulation paradigm were repeated three times on separate days (FIG. 13). Results demonstrate high consistency in the evoked response across days. Results demonstrate high consistency within person in evoked brain activity over time.

At least one technical effect of the systems and methods described herein includes (a) configurable motherboard with daughterboards for providing scalability and independent control; (b) isolated SPI interface for providing control over stimulation parameters while reducing risk of electrical interference or unintended shocks; (c) methods for monitoring and adjusting power to maintain stimulation parameters.

Some embodiments involve the use of one or more electronic processing or computing devices. As used herein, the terms “processor” and “computer” and related terms, e.g., “processing device,” and “computing device” are not limited to just those integrated circuits referred to in the art as a computer, but broadly refers to a processor, a processing device or system, a general purpose central processing unit (CPU), a graphics processing unit (GPU), a microcontroller, a microcomputer, a PLC, a RISC processor, a FPGA, a digital signal processor (DSP), an ASIC, and other programmable circuits or processing devices capable of executing the functions described herein, and these terms are used interchangeably herein. These processing devices are generally “configured” to execute functions by programming or being programmed, or by the provisioning of instructions for execution. The above examples are not intended to limit in any way the definition or meaning of the terms processor, processing device, and related terms.

The various aspects illustrated by logical blocks, modules, circuits, processes, algorithms, and algorithm steps described above may be implemented as electronic hardware, software, or combinations of both. Certain disclosed components, blocks, modules, circuits, and steps are described in terms of their functionality, illustrating the interchangeability of their implementation in electronic hardware or software. The implementation of such functionality varies among different applications given varying system architectures and design constraints. Although such implementations may vary from application to application, they do not constitute a departure from the scope of this disclosure.

Aspects of embodiments implemented in software may be implemented in program code, application software, application programming interfaces (APIs), firmware, middleware, microcode, hardware description languages (HDLs), or any combination thereof. A code segment or machine-executable instruction may represent a procedure, a function, a subprogram, a routine, a subroutine, a module, a software package, a class, or any combination of instructions, data structures, or program statements. A code segment may be coupled to, or integrated with, another code segment or an electronic hardware by passing or receiving information, data, arguments, parameters, memory contents, or memory locations. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the disclosed functions may be embodied, or stored, as one or more instructions or code on or in memory. In the embodiments described herein, memory includes non-transitory computer-readable media, which may include, but is not limited to, media such as flash memory, a random access memory (RAM), read-only memory (ROM), erasable programmable read-only memory (EPROM), electrically erasable programmable read-only memory (EEPROM), and non-volatile RAM (NVRAM). As used herein, the term “non-transitory computer-readable media” is intended to be representative of any tangible, computer-readable media, including, without limitation, non-transitory computer storage devices, including, without limitation, volatile and non-volatile media, and removable and non-removable media such as a firmware, physical and virtual storage, CD-ROM, DVD, and any other digital source such as a network, a server, cloud system, or the Internet, as well as yet to be developed digital means, with the sole exception being a transitory propagating signal. The methods described herein may be embodied as executable instructions, e.g., “software” and “firmware,” in a non-transitory computer-readable medium. As used herein, the terms “software” and “firmware” are interchangeable and include any computer program stored in memory for execution by personal computers, workstations, clients, and servers. Such instructions, when executed by a processor, configure the processor to perform at least a portion of the disclosed methods.

As used herein, an element or step recited in the singular and proceeded with the word “a” or “an” should be understood as not excluding plural elements or steps unless such exclusion is explicitly recited. Furthermore, references to “one embodiment” of the disclosure or an “exemplary” or “example” embodiment are not intended to be interpreted as excluding the existence of additional embodiments that also incorporate the recited features. Likewise, limitations associated with “one embodiment” or “an embodiment” should not be interpreted as limiting to all embodiments unless explicitly recited.

Disjunctive language such as the phrase “at least one of X, Y, or Z,” unless specifically stated otherwise, is generally intended, within the context presented, to disclose that an item, term, etc. may be either X, Y, or Z, or any combination thereof (e.g., X, Y, and/or Z). Likewise, conjunctive language such as the phrase “at least one of X, Y, and Z,” unless specifically stated otherwise, is generally intended, within the context presented, to disclose at least one of X, at least one of Y, and at least one of Z.

The disclosed systems and methods are not limited to the specific embodiments described herein. Rather, components of the systems or steps of the methods may be utilized independently and separately from other described components or steps.

This written description uses examples to disclose various embodiments, which include the best mode, to enable any person skilled in the art to practice those embodiments, including making and using any devices or systems and performing any incorporated methods. The patentable scope is defined by the claims and may include other examples that occur to those skilled in the art. Such other examples are intended to be within the scope of the claims if they have structural elements that do not differ from the literal language of the claims, or if they include equivalent structural elements with insubstantial differences form the literal language of the claims.

What is claimed is:

1. A transcranial stimulation system for delivering transcranial electric stimulation to a subject, the transcranial stimulation system comprising:
a transcranial stimulation assembly comprising:
a motherboard configured to communicate with an application installed on a stimulation computing device to receive a command of electric stimulation from the application; and
one or more daughterboards pluggable with one or more electrical connectors on the motherboard, the one or more daughterboards configured to deliver electric stimulation to a subject according to the command; and
the stimulation computing device comprising at least one processor in communication with at least one memory device, the at least one processor programmed to:
receive the command from a user, the command including at least one of a waveform, a function, or a type of the electric stimulation; and
communicate the command to a transcranial stimulation assembly.

2. The transcranial stimulation system of claim 1, wherein the transcranial stimulation assembly further comprises a plurality of daughterboards, at least one of the plurality of daughterboards configured to perform a different function from another one of the plurality of daughterboards.

3. The transcranial stimulation system of claim 1, wherein the motherboard is configured to detect that the one or more daughterboards are operating based on detection of at least one of a voltage or a current from the one or more daughterboards.

4. The transcranial stimulation system of claim 1, wherein the motherboard comprises:
a general portion; and
an isolated portion isolated from the general portion, the one or more daughterboards pluggable in the isolated portion.

5. The transcranial stimulation system of claim 1, wherein the transcranial stimulation assembly further comprises a universal serial bus (USB) connector configured to receive communication and power from the stimulation computing device via a single USB connection.

6. The transcranial stimulation system of claim 1, wherein the application is a mobile app.

7. A transcranial stimulation assembly for delivering transcranial electric stimulation to a subject, the transcranial stimulation assembly comprising:
a motherboard configured to communicate with an application installed on a stimulation computing device to receive a command of electric stimulation from the application; and
one or more daughterboards pluggable with one or more electrical connectors on the motherboard, the one or more daughterboards configured to deliver electric stimulation to a subject according to the command.

8. The transcranial stimulation assembly of claim 7, further comprising a plurality of daughterboards, wherein at least one of the plurality of daughterboards is configured to perform a different function from another one of the plurality of daughterboards.

9. The transcranial stimulation assembly of claim 7, wherein the motherboard is configured to detect that the one or more daughterboards are operating based on detection of at least one of a voltage or a current from the one or more daughterboards.

10. The transcranial stimulation assembly of claim 7, wherein the motherboard is configured to manage power consumption by the one or more daughterboards to meet one or more requirements.

11. The transcranial stimulation assembly of claim 10, wherein the one or more daughterboards are configured to detect a current usage by the one or more daughterboards and send the current usage to the motherboard, and the motherboard is configured to manage the power consumption based on the current usage.

12. The transcranial stimulation assembly of claim 7, wherein the motherboard comprises:
a general portion; and
an isolated portion isolated from the general portion, the one or more daughterboards pluggable in the isolated portion.

13. The transcranial stimulation assembly of claim 7, further comprising a universal serial bus (USB) connector configured to receive communication and power from the stimulation computing device via a single USB connection.

14. The transcranial stimulation assembly of claim 7, wherein the application is a mobile app.

15. The transcranial stimulation assembly of claim 7, wherein the motherboard is configured to communicate with the stimulation computing device via at least one of wireless communication or wired communication.

16. The transcranial stimulation assembly of claim 7, wherein the motherboard is configured to receive the command programmed by a user.

17. The transcranial stimulation assembly of claim 7, wherein the motherboard is configured to receive the command including at least one of a waveform, an amplitude, or a type of the electric stimulation.

18. One or more non-transitory machine-readable storage media for delivering transcranial electric stimulation to a subject, the one or more non-transitory machine-readable storage media comprising a plurality of instructions stored thereon that, in response to being executed, cause a system to:
receive a command of electric stimulation from a user, the command including at least one of a waveform, a function, or a type of the electric stimulation; and
communicate the command to a transcranial stimulation assembly.

19. The one or more non-transitory machine-readable storage media of claim 18, wherein the plurality of instructions further cause the system to:
communicate and power the transcranial stimulation assembly via a single universal serial bus (USB) connection.

20. The one or more non-transitory machine-readable storage media of claim 18, wherein the plurality of instructions further cause the system to:
communicate with the transcranial stimulation assembly via at least one of wireless communication or wired communication.
